SEQUENCE LISTING (17 sequences)

SEQ ID NO: 1 - protein, 127 aa, Saccharomyces cerevisiae
MSLQSNSVKPTEIPLSEIRRPLAPVLDPQKIDAMVATMKGIPTASKTCSLEQAEAAASAGELPPVDVLGVRVKGQTLYYAFGGCHRLQAYDRRARETQNAAFPVRCRVLPATPRQIRMYLGSSLDIE

SEQ ID NO: 2 - protein, 120 aa, Candida albicans
MSMYTSRLATEYVPLSEIKRPIPPVLDYQKIDAMLSTLKGVPMESATCKVEDITAGELPPIDVFKIRENGKNFYFAFGGCHRFQAYDRISKETEKEVMVKSRILPATRKSLRIYLGASVD

SEQ ID NO: 3 - protein, 124 aa, Schizosaccharomyces pombe
MTSIHTGSNNNIVELDMSELIRPIPPVLDMNKVNSMMETMTGKTPPASCGLTSEDLEAGELPPVDVLTFKKSGKPYYFAFGGCHRLRAHDEAGRKKVRCKLVNCSPNTLRLYLGASANKFLDSD

SEQ ID NO: 4 - protein, 137 aa, Homo sapiens
MGLRAGGTLGRAGAGRGAPEGPGPSGGAQGGSIHSGRIAAVHNVPLSVLIRPLPSVLDPAKVQSLVDTIREDPDSVPPIDVLWIKGAQGGDYFYSFGGCHRYAAYQQLQRETIPAKLVQSTLSDLRVYLGASTPDLQ

SEQ ID NO: 5 - protein, 136 aa, Mus musculus
MGLRAGGALRRAGAGPGAPVVHGPGGAQGGSIHSGCIATVHNVPIAVLIRPLPSVLDPAKVQSLVDTILADPDSVPPIDVLWIKGAQGGDYYYSFGGCHRYAAYQQLQRETIPAKLVRSTLSDLRMYLGASTPDLQ

SEQ ID NO: 6 - protein, 162 aa, Drosophila melanogaster
MEFISHFLRATSRRTAALGPILQRNRSEIIQKQSLTNRQAFRRYRSSCSTMDTTVHSAGIDETHLVPMSVIQRPIPSVLDEQKVQSLMETIKNETSEDEVPPIDLLWISGSEGGDYYFSFGGCHRFEAYKRLQRPTIKAKLVKSTLGDLYHYMGSSAPKYLA

SEQ ID NO: 7 - protein, 125 aa, Arabidopsis thaliana
MANLMMRLPISLRSFSVSASSSNGSPPVIGGSSGGVGPMIVELPLEKIRRPLMRTRSNDQNKVKELMDSIRQIGLQVPIDVIEVDGTYYGFSGCHRYEAHQKLGLPTIRCKIRKGTKETLRHHLR

SEQ ID NO: 8 - protein, 86 aa, Thermosynechococcus elongatus
MRVLDLPLNAIRRPLVRQTDPAKVAALMASIAEIGQQEPIDVLEVEGHYYGFSGCHRYEACQRLGLPTIRARVRRAPRSVLNLHLA

SEQ ID NO: 9 - protein, 87 aa, Nostoc sp.
MVRVQEIPLNQIRRPLPRGNDPYKVQALMESIAAIGQQEPIDVLEVDGQYYGFSGCHRYEACQRLGKETILARVRKAPRSVLKMHLA

SEQ ID NO: 10 - protein, 141 aa, Oryza sativa
MAASGFLLRCPAAPSAVPLWGRSGRGGGGGLAFSASSSNGAAVPSSLSDSEKKGPVVMEIPLDKIRRPLMRTRANDPAKVQELMDSIRVIGLQVPIDVLEVDGVYYGFSGCHRYEAHQRLGLPTIRCKVRRGTKETLRIGC

SEQ ID NO: 11 - DNA, 20 bp, Artificial Sequence
  Description of Artificial Sequence Synthetic primer
gtcccgcggcggcggcgacg

SEQ ID NO: 12 - DNA, 20 bp, Artificial Sequence
  Description of Artificial Sequence Synthetic primer
agcaggtgccaaggaggctg

SEQ ID NO: 13 - DNA, 32 bp, Artificial Sequence
  Description of Artificial Sequence Synthetic primer
ttaattgaattcatggggctgcgtgcaggagg

SEQ ID NO: 14 - DNA, 44 bp, Artificial Sequence
  Description of Artificial Sequence Synthetic primer
ttttccttttgcggccgcctactactgcaagtctggtgtggatg

SEQ ID NO: 15 - protein, 6 aa, Artificial Sequence
  Description of Artificial Sequence Synthetic peptide
FXGCHR

SEQ ID NO: 16 - protein, 6 aa, Artificial Sequence
  Description of Artificial Sequence Synthetic peptide
FSGCHR

SEQ ID NO: 17 - protein, 6 aa, Artificial Sequence
  Description of Artificial Sequence Synthetic 6xHis tag
HHHHHH

(19) United States
(12) Patent Application Publication
(10) Pub. No.: US 2011/0189154 A1
(43) Pub. Date: Aug. 4, 2011

(54) Applications For A New Class Of Enzymes: Sulfiredoxins

(76) Inventors: Michel Toledano, Boulogne-Billancourt (FR); Benoît Biteau, Les Ulis (FR)

(21) Appl. No.: 10/563,375

(22) PCT Filed: Jul. 2, 2004
(86) PCT No.: PCT/FR04/01727

§ 371 Date: May 26, 2006

(30) Foreign Application Priority Data
Jul. 4, 2003 (FR) 0308212

(51) Int. Cl. A61K 38/44; A61K 38/08; C07K 7/06

(52) U.S. Cl. 424/94.4; 530/329; 435/6.16; 514/17.7; 435/6.12

(57) ABSTRACT

Applications of a new class of enzymes, sulfiredoxines (Srx), catalyzing the reduction of Cys-SO^{2#191H} (sulfinic cystein acid) and the reduction of peroxyredoxine (Prx) in the Cys-SO2#191H form thereof into a thiol derivative.

The present invention relates to the applications of a new class of enzymes, sulfiredoxins (Srx), which catalyzes the reduction of Cys-SO₂H (cysteine-sulfinic acid) derivatives, and in particular the reduction of peroxyredoxin (Prx) in its Cys-SO₂H form to a thiol derivative.

In proteins, certain thiol groups of cysteine (Cys-SH), that have a redox activity, can be oxidized with hydrogen peroxide (H₂O₂) to sulfenic acid (Cys-SOH). Since the latter is unstable, it reacts either with any nearby thiol group so as to form a disulfide bridge (C—S—S—C), or, in the absence of an accessible nearby thiol group, the Cys-SOH compound may be further oxidized to stable sulfinic acid (Cys-SO₂H) or cysteic acid (Cys-SO₃H).

Peroxyredoxins (Prxs) are antioxidizing enzymes that contain such cysteines with redox activity. For example, the 2-Cys Prxs are inverted homodimers with 2 cysteines with redox activity per subunit. They catalyze the reduction of hydrogen peroxide.

The catalytic site of these enzymes comprises two cysteines with redox activity (N-terminal peroxydatic cysteine (Cys_{P}) and C-terminal resolving cysteine (Cys_{R}))

More specifically, the catalytic site of these peroxyredoxins comprises (Wood Z A et al., Science, 2003, 300, 650-653; Wood et al., Trends in Biochemical Sciences, 2003, 28, 1, 32-40):
the oxidation of Cys_{P}-SH to Cys_{P}-SOH (sulfenic acid) by H₂O₂;
the formation of a disulfide bridge between the Cys_{P} and the Cys_{R} of the second subunit of Prx (Cys_{P}-S—S-Cys_{R}) (slow process);
the reduction of this disulfide bridge by conventional cellular reducing agents such as glutathione or thioredoxin (Trx), so as to obtain the starting product Cys-SH.

In certain cases, Prxs can be inactivated, by superoxidation of Cys_{P}-SOH to sulfinic acid (Cys_{P}-SO₂H); this superoxidation reaction was, up until now, considered to be irreversible (Wood Z A et al., Science, 2003, 300, 650-653). Recently (Woo H A et al., Science, 2003, 300, 653-656; Georgiou G. et al., Science, 2003, 300, 592-594), the reversion of Cys-sulfinic acid to a Cys-SH compound has been shown, in vivo, in the case of mammalian two-cysteine peroxyredoxin (2-Cys Prx), indicating the existence of a specific reductase, which has not however been identified. More specifically, these authors have shown, by metabolic labeling of mammalian cells with ³⁵S, that the sulfinic form of peroxidin I, produced when cells are exposed to H₂O₂, is rapidly reduced to a catalytically active thiol form. These authors think that the reduction of sulfinic acid observed during the studies requires the intervention of specific enzymes, which have not been identified. Given that mammalian Prxs regulate H₂O₂-mediated signaling, their reversible inactivation could be used in the regulatory process.

Peroxyredoxins (Chae et al., P.N.A.S., 1994, 91, 7022-7026) are ubiquitous antioxidants which, in many species (microorganisms, plants and higher organisms, including mammals), control H₂O₂ levels, which regulate the signaling cascades leading to cell proliferation, differentiation and apoptosis (Fujii J. et al., Redox Rep., 2002, 7, 123-130).

The inventors have now identified the family of enzymes which reduce Cys_{P}-SO₂H Prxs. It involves a protein that comprises at least one catalytic site having the following motif: FXGCHR, with X=G or S, and which has a molecular weight of approximately 8 to 14 kDa.

This enzyme is conserved in eukaryotes and is hereinafter referred to as sulfiredoxin (Srx). In yeast, and in particular in Saccharomyces cerevisiae, it is referred to as Srx1 and has a molecular weight of 13 kDa. In humans, this enzyme is referred to as hSrx1 and has a molecular weight of 13.6 kDa.

Polypeptide sequences identical to those of sulfiredoxin and also the corresponding nucleotide sequences appear in the NCBI or GenBank sequence database under the following accession numbers: S. cerevisiae: YKL086W, Homo sapiens: AAH47707, CAC28314, M. musculus: BAB24939, AAH11325, Arabidopsis thaliana: AAD21682, AA042977, Oryza sativa: BAA95812, Schizosaccharomyces pombe: SPBC106.02c, Thermosynechococcus. elongatus: BAC07716, Drosophila melanogaster: AAF48773, Nostoc sp. (PCC7120): NP_{—}488186.

On the other hand, no function has been attributed to these polypeptide sequences, in the NCBI or GenBank sequence database.

The inventors have now found a common point between these various proteins: the abovementioned catalytic site and a function: catalysis of the reduction of Cys_{P}-SO₂H Prxs.

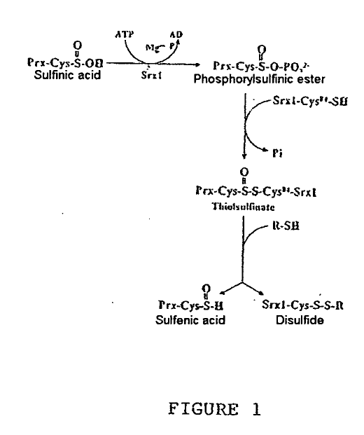

The reaction catalyzed by sulfiredoxin (Srx) is summarized in FIG. 1.

Consequently, a subject of the present invention is the use of a protein called sulfiredoxin (Srx), which comprises at least one catalytic site having the following motif: FXGCHR, with X=G or S, for catalyzing the reduction of peroxyredoxins (Prxs) in their superoxide form Prx-Cys_{P}-SO₂H (peroxyredoxin cysteine sulfinic acid) to a thiol derivative (SH).

Sulfiredoxin therefore plays a very important role in the antioxidizing function of peroxyredoxins and is involved in the repair or the control of proteins modified by the formation of a cysteine-sulfinic acid.

According to an advantageous embodiment of said use, said sulfiredoxin is a sulfiredoxin of a microorganism, a plant or a higher organism, which generally comprises between 80 and 170 amino acids and at least the catalytic site having the following motif: FXGCHR, with X=G or S. They have the following percentage identities and similarities with respect to one another:
yeast/human: 32% identity and 67% similarity
yeast/plants: 23% identity and 39% similarity
yeast/mouse: 31% identity and 51% similarity
yeast/fungi: 80% identity and 90% similarity.

In accordance with the invention, the identity of a sequence compared with a reference sequence (SEQ ID No. 1 corresponding to the sequence of S. cerevisiae Srxl) is assessed as a function of the percentage of amino acid residues that are identical, when the sequences corresponding to the catalytic region as defined above are aligned, so as to obtain the maximum correspondence between them.

A protein having an amino acid sequence having at least X % identity with the reference sequence SEQ ID No. 1 is defined, in the present invention, as a protein that may include up to 100-X alterations per 100 amino acids of the sequence SEQ ID No. 1. For the purpose of the present invention, the term “alteration” includes consecutive or dispersed deletions, substitutions or insertions of amino acids into the reference sequence. This definition applies, by analogy, to the nucleic acid molecules.

The similarity of a sequence compared with the reference sequence SEQ ID No. 1 is assessed as a function of the percentage of amino acid residues that are identical or that differ in terms of conservative substitutions, when the sequences are aligned so as to obtain the maximum correspondence between them. For the purpose of the present invention, the term “conservative substitution” is intended to mean the substitution of one amino acid with another that has similar chemical properties (size, charge or polarity), and that generally does not modify the functional properties of the protein.

A protein having an amino acid sequence having at least X % similarity with the sequence SEQ ID No. 1 is defined, in the present invention, as a protein whose sequence may include up to 100-X nonconservative alterations per 100 amino acids of the reference sequence. For the purpose of the present invention, the term “nonconservative alterations” includes deletions, nonconservative substitutions or consecutive or dispersed insertions of amino acids in the sequence SEQ ID No. 1.

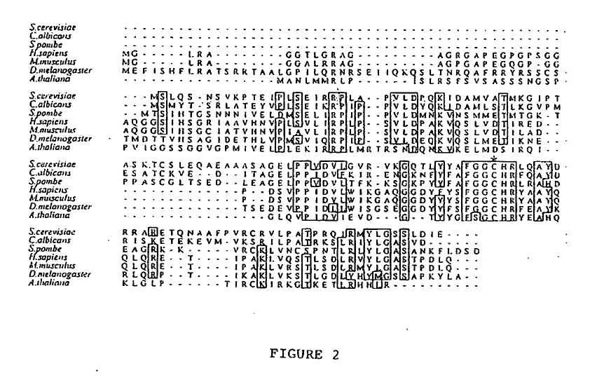

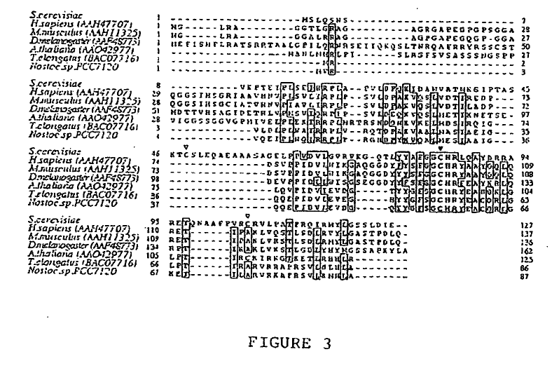

Said sulfiredoxin is in particular selected from proteins whose sequences correspond, respectively, to the sequences SEQ ID Nos. 1 to 10, illustrated in FIGS. 2 and 3 or represented in the sequence listing: S. cerevisiae: SEQ ID No. 1; C. albicans: SEQ ID No. 2; S. pombe: SEQ ID No. 3; H. sapiens: SEQ ID No. 4; M. musculus: SEQ ID No. 5; D. melanogaster: SEQ ID No. 6; A. thaliana: SEQ ID No. 7; T. elongatus: SEQ ID No. 8; Nostoc sp.: SEQ ID No. 9 and Oryza sativa: SEQ ID No. 10.

A subject of the present invention is also an isolated peptide corresponding to the catalytic site of Srx, characterized in that it is defined by the following sequence: FXGCHR, with X═S.

A subject of the present invention is also anti-Srx antibodies, characterized in that they are obtained by suitable immunization of an animal with an Srx protein, defined by a sequence selected from the group consisting of SEQ ID NOS: 1-3, 5-6 and 8-10, or the peptide FXGCHR, with X═S.

Said antibodies are either polyclonal antibodies or monoclonal antibodies.

A subject of the present invention is also a medicinal product, characterized in that it comprises an effective amount of a protein defined by a sequence selected from the group consisting of SEQ ID Nos. 1-3 and 5-10, and, optionally, at least one pharmaceutically acceptable excipient.

A subject of the present invention is also the use of a protein as defined above, for preparing an antioxidizing medicinal product for use in the treatment of cancers, neurodegenerative disorders and neuromuscular diseases, in which a fault in the Prx/Srx antioxidizing system is observed.

A subject of the present invention is also a method of screening for diseases related to cancer, to aging, to neurodegenerative diseases and to neuromuscular diseases, which method is characterized in that it comprises, for evaluating the involvement of the Prx/Srx antioxidizing system:
(1) bringing the cells of a biological sample into contact, in vitro, with hydrogen peroxide (H₂O₂),
(2) detecting the Prx-Cys_{P}-SO₂H formed, between 1 hour and 4 hours after said bringing into contact according to step (1), and
(3) establishing the ratio of the amounts of Prx-Cys_{P}-SO₂H and of Prx-Cys_{P}-SH, from 4 hours after said bringing into contact according to step (1).

The biological sample consists in particular of blood cells.

Prx-Cys_{P}-SO₂H/Prx-Cys_{P}-SH ratios >1 are the sign of a Prx/Srx antioxidizing system pathology related to a dysfunction of Srx.

Thus, such a method makes it possible to evaluate whether or not the Prx/Srx antioxidizing system is functioning normally. Knowledge of the mechanisms involved in the etiology of the disease makes it possible to select the treatment most suited to the situation, in particular in the case of faulty Prx/Srx antioxidizing systems.

As variants, said screening method comprises:

A. genotyping of the sulfiredoxin, using the total RNA of a suitable biological sample, in particular blood cells.

More specifically, said method comprises:

(1) extracting the total RNA from a suitable biological sample,

(2) preparing specific sulfiredoxin cDNA by amplification of the RNA using the following two primers:

| | |
|---|---|
| GTCCCGCGGCGGCGGCGACG | (SEQ ID No. 11) |
| AGCAGGTGCCAAGGAGGCTG, | (SEQ ID No. 12 ) |

these sequences being located, respectively, upstream and downstream of the human sulfiredoxin ORF (GenBank No. AAH47707),

(3) establishing its nucleotide sequence, and

(4) comparing with respect to a DNA sequence encoding an Srx protein, as defined above, derived from the same species as that of the biological sample to be analyzed.

B. relative quantification, by any appropriate means, of the mRNA encoding human sulfiredoxin (hSrx1) from the total cDNA prepared from a human biological sample, by comparison with a reference sample.

The reference sample is in particular a sample obtained from a normal control individual.

In accordance with the invention, prior to said quantification, said method comprises a total RNA extraction step.

According to an advantageous arrangement of this embodiment, said quantification comprises:

(al) preparing cDNA from the total RNA by reverse transcription with appropriate primers, and in particular random hexanucleotide primers;

(a2) amplifying said cDNA in the presence of the pair of primers:

| | |
|---|---|
| GTCCCGCGGCGGCGGCGACG | (SEQ ID No. 11) |
| AGCAGGTGCCAAGGAGGCTG, | (SEQ ID No. 12) |

in the presence of a fluorescent reporter, and simultaneously or sequentially,

(a3) detecting the amount of the amplimer (or amplicon) by measuring the fluorescent signal.

The mRNA amplification is carried out by RT-PCR; the reverse transcription and PCR amplification steps are either separate, and in this case the quantification is carried out by quantitative PCR, or they are coupled, and in this case, the quantification is carried out by quantitative RT-PCR.

Preferably, said quantification is carried out using an internal standard such as, for example, the 18S ribosomal RNA subunit.

According to another advantageous arrangement of this embodiment, the fluorescent reporter is selected from the group consisting of agents that bind to double-stranded DNA and fluorescent probes.

Preferably, said quantification is carried out in real time, i.e. the detection and the quantification of the fluorescent signal emitted are carried out during the amplification process, insofar as the increase in signal is directly proportional to the amount of amplimers produced during the reaction.

The general principles of real-time quantitative PCR and RT-PCR, and also the various techniques for the quantitative detection of ampliers: using agents that bind to double-stranded DNA (intercalating agents: ethidium bromide, SYBR Green I, YO-PRO-1; agents that bind to the minor groove: Hoechst 33258) or using fluorescent probes, i.e.: hydrolysis of probes by the 5′ nuclease activity of DNA polymerase (TaqMan™), hybridization of 2 probes (Hybprobes), molecular beacons and scorpion primers, are known to those skilled in the art and they are in particular described in Poitras et al., Reviews in Biology and Biotechnology, 2002, 2, 1-11. The real-time quantitative PCR and RT-PCR using probes of the TaqMan™ type are in particular described, respectively, in Heid C. et al. (Genome Research, 1996, 6, 986-994) and Gibson U. et al. (Genome Research, 1996, 6, 995-1001).

According to an advantageous mode of this embodiment, when said fluorescent reporter is a probe, it is preferably selected from the group consisting of the probes defined by the following sequences:

(SEQ ID No. 13)
TTAATTGAATTCATGGGGCTGCGTGCAGGAGG
and
(SEQ ID No. 14)
TTTTCCTTTTGCGGCCGCCTACTACTGCAAGTCTGGTGTGGATG.

The RNA extraction, the cDNA preparation and the establishment of the sequence are carried out using conventional techniques, according to standard protocols such as those described in Current Protocols in Molecular Biology (Frederick M. AUSUBEL, 2000, Wiley and Son Inc., Library of Congress, USA).

A subject of the present invention is also a method of screening for diseases related to cancer, to aging, to neurodegenerative diseases and to neuromuscular diseases, which method is characterized in that it comprises:
immunodetection of the Srx protein in a biological sample to be tested, using an antibody obtained by suitable immunization of an animal with an Srx protein or the peptide FXGCHR, with X=G or S, after separation of total proteins by electrophoresis, then
evaluation of the quality and of the amount of said Srx protein compared with a control Srx protein.

Said detection-quantification is advantageously carried out by the Western blotting method.

A subject of the present invention is also the use of the sequence encoding an Srx protein, as defined above, or of a vector containing said coding sequence, for obtaining plants whose abilities to withstand stress (drought, cold, heat, oxidizing toxic agents present in the environment) are significantly increased.

The sequences encoding the Srx protein can be readily obtained from the abovementioned sequence databases.

A subject of the present invention is also host cells, characterized in that they are transformed with a recombinant vector containing a sequence encoding an Srx protein, defined by a sequence selected from the group consisting of SEQ ID No: 1-3, 5-6 and 8-10.

According to an advantageous embodiment of said host cell, it consists of an S. cerevisiae strain overexpressing the SRX1 gene.

According to another advantageous embodiment of said host cell, it consists of a mammalian cell modified with a vector overexpressing the hSrx1 gene.

The vector is advantageously an E. coli/S. cerevisiae shuttle vector comprising, at a cloning site, the sequence encoding the Srx protein and the promoter of the Srx gene. It is in particular the plasmid pRS316 (ATCC No. 77145).

The promoter of the Srx gene is 400 base pairs upstream of the translation initiation site; it can be found on the site http://www.yeastgenome.org/(accession No. YKL086W).

These host cells transformed with such a vector are particularly advantageous for studying the Prx/Srx antioxidizing system and screening, in vitro, for medicinal products that modulate the activity of the Prx/Srx antioxidizing system.

Consequently, a subject of the present invention is also a method of screening for medicinal products capable of modulating the activity of the Prx/Srx antioxidizing system, characterized in that it comprises:

(1) bringing the substance to be screened into contact with host cells according to the invention, in the presence of hydrogen peroxide,

(2) detecting the Prx-Cys_{P}-SO₂H formed, between 1 hour and 4 hours after said bringing into contact according to step (1),

(3) establishing the ratio of the amounts of Prx-Cys_{P}-SO₂H and of Prx-Cys_{P}-SH, from 4 hours after said bringing into contact according to step (1).

A subject of the present invention is also a method of screening for medicinal products that are useful in the treatment of cancers, of neurodegenerative diseases and of neuromuscular diseases, related to a fault in the Prx/Srx antioxidizing system, characterized in that it comprises:

a) bringing the substance to be tested into contact with an extract of modified host cells as defined above or a biological sample of a nonhuman transgenic animal, in particular mice, selected from the group consisting of animals in which the gene of the Srx protein is knocked out and animals in which the gene of the Srx protein is overexpressed, in the presence of hydrogen peroxide,

b) measuring, by any appropriate means, the antioxidizing activity of the Prx/Srx system of the mixture obtained in a), and

c) selecting the substances capable of stimulating or of inhibiting said activity.

The measurement of said activity is in particular carried out by detecting the Prx-Cys_{P}-SO₂H formed, between 1 hour and 4 hours after said bringing into contact according to step (a), and establishing the ratio of the amounts of Prx-Cys_{P}-SO₂H and of Prx-Cys_{P}-SH, from 4 hours after said bringing into contact according to step (a).

A subject of the present invention is also a method of screening for medicinal products that are useful in the treatment of cancers, of neurodegenerative diseases and of neuromuscular diseases, related to a fault in the Prx/Srx antioxidizing system, characterized in that it comprises:

(1) bringing the substance to be screened into contact with nonhuman transgenic mammals, in particular mice, selected from the group consisting of animals in which the gene of the Srx protein is knocked out and animals in which the gene of the Srx protein is overexpressed, and

(2) measuring the survival of the animal.

The production of nonhuman transgenic mammals is carried out using conventional methods, and in particular according to the protocols described in Transgenic animals generation and use (C. M. Houdebine Ed., Harwood academic publishers, Amsterdam).

A subject of the present invention is also a method of reducing a product comprising at least two cysteines with redox activity, which method is characterized in that it comprises bringing said protein into contact with a sulfiredoxin (Srx), which comprises at least one catalytic site having the following motif: FXGCHR, with X=G or S, in the presence of ATP and of magnesium.

The reduction of the product comprising at least two cysteines with redox activity involves its activation by phosphorylation, followed by reduction of the sulfur, these two activities being catalyzed by sulfiredoxin.

A subject of the present invention is also a method of synthesizing a product comprising Cys-SH residues from products comprising Cys-SO₂H residues, characterized in that it comprises a step consisting of reduction of the product comprising the Cys-SO₂H residues to a product comprising Cys-SH residues, in the presence of a sulfiredoxin, of ATP and of magnesium.

Besides the above arrangements, the invention also comprises other arrangements, which will emerge from the description that follows, that refers to examples of implementation of the method that is the subject of the present invention and also to the attached drawings, in which:

FIG. 1 illustrates the reaction catalyzed by Srx1;

FIGS. 2 and 3 represent the comparison of the Srx1 sequences in various species; FIG. 2: S. cerevisiae, C. albicans, S. pombe, H. sapiens, M. musculus, D. melanogaster and A. thaliana; the identical regions are boxed in; the catalytic site is located around the conserved cysteine, indicated by an asterisk; FIG. 3: S. cerevisiae, H. sapiens, M. musculus, D. melanogaster, A. thaliana, T. elongatus and Nostoc sp. The GenBank accession Nos. are indicated on this figure. The sequence alignment was carried out using the CLUSTALW program. The amino acids that are identical in approximately 65% of the sequences are boxed in. The Srx1 active site comprising a cysteine (black arrow) and the other cysteines (white arrow) are indicated;

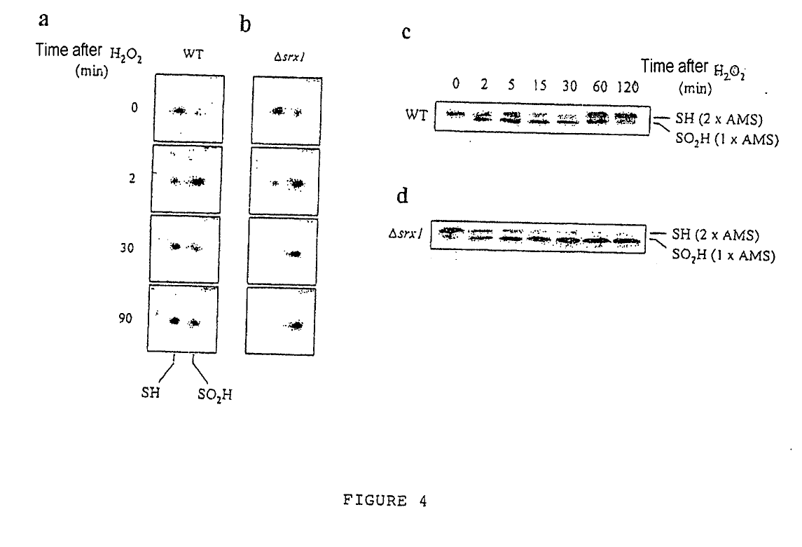

FIG. 4 illustrates the recycling of the cysteine-sulfinic acid form of Tsa1, which is dependent on Srx1; FIGS. 4a and 4b: 2-D PAGE analysis of the reduced (SH) and oxidizing (SO₂H) forms of Tsa1 labeled with ³⁵S-Met in wild-type cells and Δsrx1 cells exposed to H₂O₂ (500 μM) for the period indicated; FIGS. 4c and 4d correspond to Western blots of reduced (2× AMS) and oxidized (1× AMS) forms of Tsa1 from WT cells (c) or from Δsrx1 cells (d) treated with H₂O₂ after alkylation in vitro with AMS. After induction of Srx1 expression for 15 min with H₂O₂ (100 μM), the cells are treated with cycloheximide (CHX) for 5 min before the treatment with H₂O₂ (500 μM);

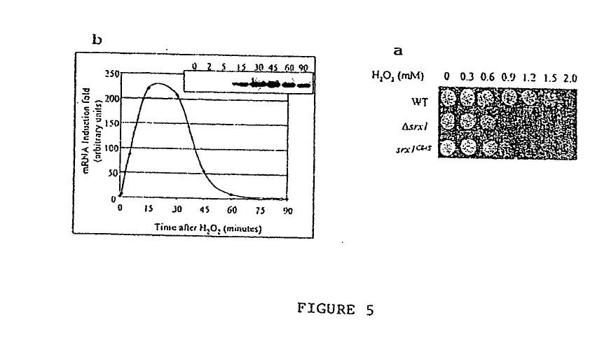

FIG. 5 illustrates the role played by the Srx1 protein in the resistance of cells to stress induced by hydrogen peroxide; sensitivity tests are carried out by growing a wild-type strain (WT) and a knockout cell (Δsrx1) or a mutant strain srx1^{C84S} in Petri dishes containing increasing concentrations (in mM) of hydrogen peroxide (H₂O₂) (FIGS. 5a and 5b) : FIG. 5a: resistance to H₂O₂ of the wild-type strain (WT), of the knockout strain (Δsrx1) and of the mutant strain srx1^{C84S}; FIG. 5b: Western blotting (inset) and QT-RT PCR of the Srx1 protein tagged with HA and of the mRNA in cells treated with hydrogen peroxide (400 μM);

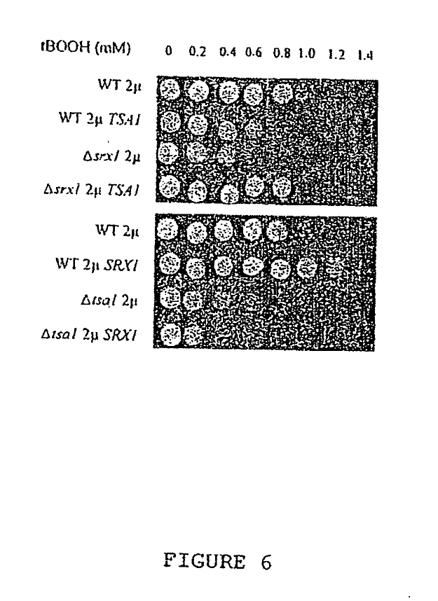

FIG. 6 illustrates the role played by the Srx1 protein in the resistance of cells to stress induced by t-butyl hydroperoxide; sensitivity tests are carried out by growing a wild-type strain (WT), a knockout cell (Δsrx1), a wild-type strain overexpressing Tsa1 or Srx1, a knockout cell (Δsrx1) expressing Tsa1, a knockout cell (Δtsa1) and a knockout cell (Δtsa1) overexpressing Srx1 in Petri dishes containing increasing concentrations of t-butyl hydroperoxide (tBOOH); the concentrations are expressed in mM;

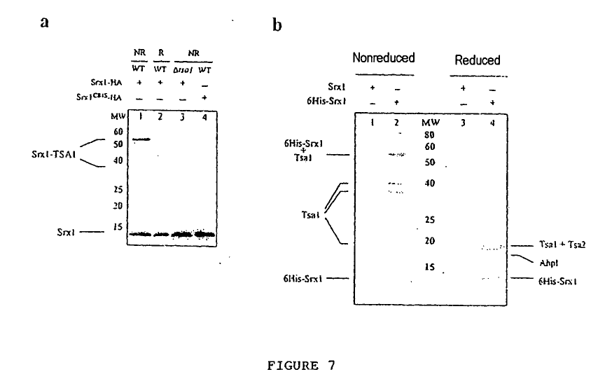

FIG. 7 illustrates the interaction between Tsa1 and Srx1 in a covalent (disulfide bridge) and noncovalent manner; FIG. 7a: Western blotting of the HA-tagged Srx1 protein (lanes 1, 2 and 3) or of HA-tagged Srx1^{C84S} (lane 4) expressed in a wild-type strain (WT) (lanes 1, 2, 4) or in Δtsa1 cells (lane 3) treated for 15 min with H₂O₂ (500 μM), after SDS-PAGE electrophoresis carried out under reducing (R) (lane 2) or nonreducing (NR) (lanes 1, 3, 4) conditions; FIG. 7b: the proteins copurified with the Srx1 tagged with 6His (lanes 2, 4) or the untagged Srx1 (lanes 1, 3) under nonreducing conditions are separated by SDS-PAGE under nonreducing (lanes 1, 2) or reducing (lanes 3, 4) conditions and visualized by Coomassie blue staining. The protein bands are identified by MALDI-TOF mass spectrometry as indicated;

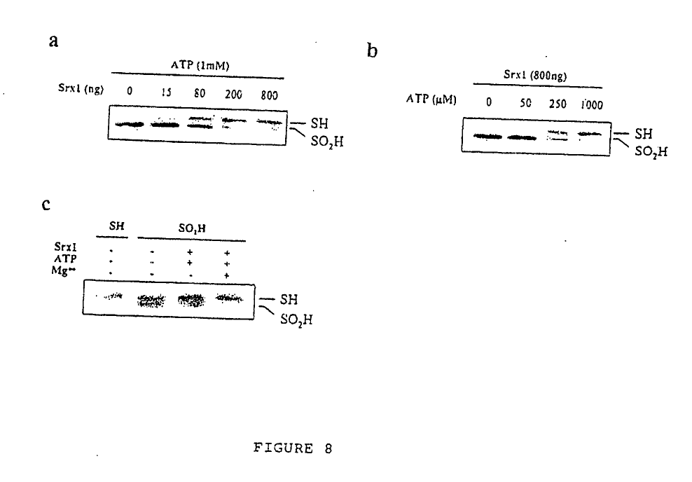

FIG. 8 shows that the Srx1 protein and ATP are required for the reduction of oxidized Tsa1 in vitro by Srx1; FIGS. 8a and b: Western blotting analysis of the reduced (SH) and superoxidized (SO₂H) forms of Myc-Tsa1 in Δtsa1 cell lysates incubated for 15 min at 30° C. with purified Srx1 and ATP, at the concentrations indicated; FIG. 8c: Western blotting analysis of the reduced (SH) and superoxidized (SO₂H) forms of 6His-Tsa1 incubated for 15 min at 30° C. with purified Srx1, ATP (1 mM) and Mg⁺⁺ (1 mM), as indicated;

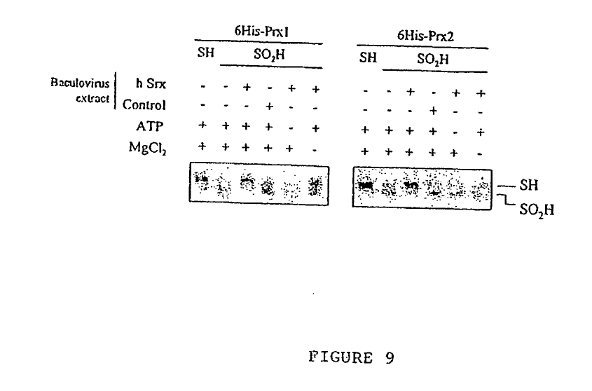

FIG. 9 illustrates the role of hSrx1 in the reduction of 6His-Prx1 and 6His-Prx2 in their superoxidized forms.

It should be clearly understood, however, that these examples are given only by way of illustration of the subject of the invention, of which they in no way constitute a limitation.

### EXAMPLE 1

### Materials and Methods

1.1. Strains

The S. cerevisiae strains used are the YPH98 strain (Sikorski R. et al., Genetics, 1989, 122, 19-27 (MATa, ura3-52, lys2-801^{amber}, ade2-101^{ochre} trp1-Δ1 leu2-Δ1) and its isogenic derivatives. The Δsrx1, Δtrr1 and Δtsa1 strains are produced by replacing the coding region of SRX1 (sulfiredoxin) and of TRR1 (thioredoxin reductase) with KANMX4, and the TSA1 open reading frame with TRP1 (tyrosinase-related protein 1).

The strains overexpressing Tsa1 and Srx1 are identical to the previous strains, except that they each carry a deletion of the Tsa1 or Srx1 gene and carry the multicopy plasmid psRS426 (NO. ATCC 77107).

The cells are cultured at 30° C. in a YPD medium (1% yeast extract, 2% bactopeptone and 2% glucose) or a CASA medium (0.67% yeast nitrogenous base, 0.1% casamino acids, 2% glucose), supplemented with adenine, tryptophan and uracil.

1.2. Plasmids

The following fusion proteins:
Srx1-HA: fusion protein comprising the fusion of two HA epitopes at the C-terminal of Srx1 and
6His-Srx1: protein from fusion between Srx1 and, at its N-terminal end, six histidine tags,

are constructed by PCR in two steps: the nucleotide primers used for the PCR incorporate the sequence of one or other of the HA epitopes (defined by the commercial antibody recognizing the HA epitope 12CA5, Babco, MMS-101 R) and 6His (6 histidines) and amplify the complete coding sequence of Srx1, flanked by 400 and 200 base pairs upstream and downstream of their sequence and cloned at the EcoRI site of the plasmid pRS316 (No. ATCC 77145) or of the plasmid pRS426 (No. ATCC 77107).

Myc-Tsa1, a fusion protein comprising, at the N-terminal end of Tsa1, a Myc epitope (defined by the anti-Myc antibody, 9E10, Babco, MMS-150R), is constructed and cloned similarly at the EcoRI site of the plasmid pRS316. The site-directed mutagenesis for the generation of the Cys>Ser mutants is carried out by a standard PCR amplification protocol using primer oligonucleotides containing the modified sequence.

1.3 Protein Analysis
For the 2-D PAGE analysis, the cell cultures at the beginning of the exponential phase (OD_{600 nm}=0.3) are labeled with ³⁵S-Met (100 μCi) for 20 min at 30° C., followed by chasing of the labeled methionine with cold methionine (final concentration of 1 mM) and cysteine (final concentration of 0.1 mM), and treated with H₂O₂ (500 μM). The cells are subjected to a 2-D PAGE analysis as described in Maillet et al. (J. Biol. Chem., 1996, 271, 10263-10270).
For the analysis of the in vivo redox state of Srx1-HA, the lysates of cell cultures at the beginning of the exponential culture phase (OD_{600 nm}=0.3) are prepared by the trichloroacetic acid lysis protocol (Delaunay et al., EMBO J., 2000, 19, 5157-5166). The precipitated proteins are solubilized in a buffer A [Tris-Cl, pH 8 (100 mM), SDS (1%), EDTA (1 mM)] containing N-ethyl-maleimide (NEM) (50 Mm).

The extracts are separated by SDS-17% PAGE under reducing and nonreducing conditions and the Srx1-HA is detected using the abovementioned monoclonal antibody 12CA5.
For the derivatization of the cysteine of Myc-Tsa1 with AMS, the cell extracts are treated under the same conditions as those of the TCA lysis protocol, except that the precipitated proteins are first solubilized in the buffer A containing DTT (50 mM) for 1 h at 37° C., precipitated with TCA, and suspended in a buffer A containing AMS (15 mM) for 2 h at 37° C. The cell extracts are separated by SDS-20% PAGE under reducing conditions and Myc-Tsa1 is immunodetected with the abovementioned anti-Myc monoclonal antibody 9E10.
For the in vitro reduction, either 3 μl of lysate (2 mg/ml) of Δsrx1 cells treated with H₂O₂ comprising oxidized Myc-Tsa1, or oxidized and purified 6His-Tsa1 (0.5 mg), are added to the reaction buffer (RM) (final volume of 80 μl) [Tris-Cl, pH 6.8 (50 mM), KCl (100 mM)] containing purified Srx1 expressed by a baculovirus, ATP and MgCl₂ at the concentrations indicated, and incubated for 15 minutes at 30° C. The 6His-Tsa1 is oxidized to cysteine-sulfinic acid by incubation in the RM buffer containing DTT (10 mM) and H₂O₂ (1 mM) for 30 min, and diluted 16 times the reaction medium.

1.4 Purification of Recombinant Proteins

Srx1 and hSrx1 are expressed in High Five insect cells using the Bac-To-Bac® baculovirus expression system (Invitrogen) and purified successively by ion exchange chromatography, affinity chromatography and HPLC (8 ml-Poros® 50HS, 8 ml-Poros® 50HE, 0.8 ml-Poros® 20HS) (Applied Biosystems).

6His-Tsa1 is expressed in E. coli BL21 cells from the plasmid pET28a-Tsa1 after induction with isopropylthio-β-D-galactopyranoside, in accordance with the manufacturer's recommendations (Stratagene). The cells are suspended in a lysis buffer [Tris-Cl, pH 6.8 (50 mM), KCl (100 mM), DTT (2 mM), imidazole (20 mM)], supplemented with phenylmethanesulfonyl fluoride (PMSF) (1 mM), and lysed by means of freezing-thawing cycles and sonication. The extracts are centrifuged for 30 min at 30 000 g and the supernatant is passed over a Ni-NTA agarose column (Qiagen). After washing of the column with the lysis buffer, the Tsa1 is eluted with lysis buffer supplemented with imidazole (150 mM).

The purity and the concentration of the purified proteins is determined by Coomassie blue staining after SDS-PAGE and the Bradford test (Biorad).

1.5 Purification of the Srx1 Reaction Partners

6His-Srx1 and Srx1 are expressed from the plasmid pRS426 in the Δtrr1 strain, devoid of the thioredoxin reductase gene which stabilizes disulfide bridges. The cells are cultured as far as the middle of the exponential phase (OD_{600 nm}=0.8) and treated with H₂O₂ (5 mM) for 5 min, washed twice in water supplemented with NEM (10 mM), frozen and lysed in an Eaton press in a buffer C [Tris-Cl, pH 8 (100 mM), NaCl (50 mM) EDTA-without protease inhibitor (Roche-Boerhinger), PMSF (1 mM), imidazole (20 mM), NEM (10 mM)]. The cell extract is centrifuged for 1 h 30 min at 10 000 g and the supernatant is passed over a Ni-NTA column (Qiagen). After washing of the column with a buffer D [Tris-Cl, pH 8 (100 mM), NaCl (50 mM)]+imidazole (20 mM), the proteins are eluted with the buffer D+imidazole (30 mM).

1.6 RNA Analysis

The total RNA is extracted as described in Lee et al. (J. Biol. Chem., 1999, 274, 4537-4544) and the cDNA is synthesized by reverse transcription with random hexanucleotide primers, using 1 μg of total RNA.

A quantitative PCR (Biorad iCycler) is carried out using the SYBR Green I fluorescent method, with the primers specific for SRX1 or ACT1, three separate times, in accordance with the supplier's recommendations.

### EXAMPLE 2

### Reversibility of the Superoxidation of the Cysteine of Tsa1 by the Catalytic Activity of Srx1

2.1 Materials and Methods

One of the 5 Prxs of S. cerevisiae, the Tsa1, is a 2-Cys Prx and constitutes the main antioxidant in yeast with a broad substrate specificity toward both H₂O₂ and organic peroxides.

The oxidation of Tsa1 and the reversibility of this reaction in the presence of Srx were analyzed according to two techniques:

(A) two-dimensional gel separation according to the isoelectric point of the protein (2-D PAGE electrophoresis); the wild-type strain cells (WT) and the Δsrx1 knockout strain are initially subjected to radioactive labeling, in vivo, of the proteins, followed by chasing of the radioactive element, before being treated with H₂O₂, for different periods (0, 2, 30 and 90 minutes of treatment); the left spot (FIG. 4a) represents the native form of the protein and the right spot (FIG. 4a) represents the acid form (sulfinic acid);

(B) differential thiol alkylation; the wild-type strain cells (WT) and the Δsrx1 knockout strain cells carrying a tagged copy of Tsa1 are treated with cycloheximide (CHX) in order to block de novo protein synthesis during analysis, and then treated with H₂O₂. The proteins are extracted, and reduced with DTT, and the thiols are then alkylated with a 500 Da compound, 4-acetamido-4′-maleimidylstilbene-2,2′-disulfonic acid (AMS) which alkylates cysteines at the level of the free SH groups but not in sulfinate form, increasing the molecular weight of the protein by 0.5 kDa per cysteine alkylated (AMS); the difference in size between the proteins carrying two alkylated thiols (reduced cysteines or disulfide bridge, indicated “2 AMS” in FIGS. 4c and 4d) or one alkylated thiol (sulfinic acid, indicated “1 AMS” in FIGS. 4c and 4d) is observed after separation according to their size on an SDS-PAGE gel. The protein is visualized by Western blotting.

2.2 Results

The results are given in FIGS. 4a and 4b.

In the nontreated cell extracts, Tsa1 appears as a double spot: one of which is intense, corresponding to approximately 85% of the total enzyme at a pI position of 4.8 (+/−0.05), which corresponds to a reduced Tsa1 (the theoretical pI of Tsa is 4.87); the other, finer spot being located at a more acidic pI position of 4.7 (+/−0.05), which corresponds to the oxidized Tsa1 (the theoretical value of the sulfinic acid form of the cystine of Tsa1 is 4.75). After treatment for 2 minutes with H₂O₂ (500 μM), the proportion of oxidized Tsa1 increases to the detriment of the reduced Tsa1, up to a proportion of approximately 90% of the total proteins. After treatment for 30 minutes, the reduced Tsa1/oxidized Tsa1 ratio returns to that of the untreated cells. The reappearance of the reduced Tsa1 spot comes from the oxidized Tsa1 and not from Tsa1 synthesized de novo, given that the protein labeling is interrupted before the analysis. Identical results are observed when the cells are treated with t-butyl hydroperoxide (t-BOOH).

In the cell extracts not treated with H₂O₂, the Tsa1 is to a large extent reduced and migrates as a double band modified by AMS (FIGS. 4c and 4d); and 15 minutes after treatment with H₂O₂, the Tsa1 migrates as single or double species modified by AMS, exhibiting a mixture of reduced and oxidized forms according to a ratio of approximately 1:3. After a period of 120 minutes of this treatment, the Tsa1 has completely returned to its initial state, i.e. in the form of doublet alkylated by AMS, demonstrating the reduction of the sulfonate to Cys-SH. The reduction of the Tsa1 is different compared to that observed by 2-D PAGE (FIG. 4a), which is probably due to the inhibition of the protein synthesis.

These two experiments show that the superoxidized form of Tsa1 (sulfinic acid) can be reduced to free thiol in a wild-type strain, and that the presence of Srx1 is essential for this reduction.

### EXAMPLE 3

### Identification of a 13 kDa Protein in S. cerevisiae Linked to a Prx Via a Disulfide Bridge (FIG. 7)

3.1. Materials and Methods (See Example 1)

(A) Cells containing a tagged (HA) copy of the Srx1 protein are treated with 500 μM of H₂O₂ for 15 minutes. The proteins are extracted according to a method that allows the intracellular redox state of the thiols to be conserved (see example 1), and then separated on an SDS-PAGE gel under reducing conditions for the cells of the wild-type strain (WT) containing a tagged (HA) copy of the Srx1 protein (lane 1) and under nonreducing conditions for the wild-type strain cells (WT) (lane 2), the Δtsa1 mutant strain carrying a tagged copy of the SRX1 gene (lane 3), and the Δsrx1 strain carrying a tagged copy of the SRX1 gene having undergone a mutation C84S (lane 4); the reference molecular weights (MW) are expressed in kDa.

(B) the Srx1 protein is purified under native conditions by means of a 6His tag, from Δtrr1 cells treated for 5 minutes with 5 mM of H₂O₂; the purified proteins are then separated on a reducing or nonreducing SDS-PAGE gel. The various proteins indicated were identified by mass spectrometry; the purified proteins separated under nonreducing and reducing conditions come from the Δtrr1 mutant strain containing a copy of the SRX1 gene (wells No. 1 and 3), and from the Δtrr1 mutant strain containing a tagged (HA) copy of the SRX1 gene (wells No. 2 and 4); the reference molecular weights (MW) are expressed in kDa.

3.2 Results

FIG. 7a demonstrates the existence of an intermolecular disulfide bridge between Tsa1 and Srx1, involving the conserved cysteine (Cys84) of Srx1 (see FIGS. 2 and 3).

It also shows that Srx1 can be in two forms: a 13 kDa monomer and a disulfide bridge-linked 55 kDa multimer (FIG. 7a, lane 2).

FIG. 7b illustrates the fact that the copurification of Tsa1, Tsa2 and Ahp1 shows that Srx1 interacts with three of the five peroxyredoxins that exist in yeast and that the interaction with Tsa1 may be redox or noncovalent.

More specifically, the purified nonreduced material contains several major bands of sizes 80, 55, 40, 35, 20 and 13 kDa (FIG. 7b), which are limited to 2 main bands of 13 and 20 kDa and a minor band of 18 kDa after reduction (last well). MALDI-TOF mass spectrometry applied to the reduced material made it possible to identify the Srx1 and Tsa1 proteins and the Ahp1 protein, which is the second major 2-Cys Prx of yeast, in the bands of 13, 20 and 18 kDa, respectively. Tsa2, which is a third 2-Cys Prx, is also present in trace form in the 20 kDa band. Mass spectrometry analysis of nonreduced lysate made it possible to identify both the Tsa1 protein and the Srx1 protein in the 55 kDa band, probably in the form of disulfide bridge-linked heterotrimers containing 2 molecules of Tsa1. This analysis also made it possible to detect the presence of the Tsa1 protein in the 40, 35 and 20 kDa bands, probably in the form of disulfide bridge-linked dimers and monomers. The association of the Srx1 and Tsa1 proteins, which are disulfide bridge-linked, is confirmed by immunodetection during which the 55 kDa band containing the Srx1 protein is not detected in the H₂O₂-treated lysates from the Δtsa1 strain devoid of the TSA1 gene. These results show that Srx1 is greatly induced by H₂O₂ and associates with Tsa1 noncovalently in the form of disulfide bridge-linked heteromers.

The Srx1 protein also associates with 2 other Prxs: Ahp1 and Tsa2, but its association is minor under the conditions tested.

### EXAMPLE 4

### Srx1 Function is Linked to Peroxidase Activity and to Tsa1

4.1 Materials and Methods

4.1.1 Materials

The wild-type strain and the two mutant strains Δtsa1 and Δsrx1 are those already described in example 1.

4.1.2 Methods

The tests for sensitivity of the wild-type and mutant strains to t-BOOH and to H₂O₂ are carried out as follows (see also example 1):
Test for sensitivity to tBOOH or to H₂O₂

Wild-type cells or cells with a knockout for the SRX1 gene are deposited onto Petri dishes containing increasing concentrations of hydrogen peroxide (H₂O₂) or of t-butyl hydroperoxide (tBOOH). The growth of the cells is observed after incubation for 48 hours at 30° C.
Extraction of proteins while at the same time preserving their cellular redox state (see example 1).

4.2. Results

FIGS. 5a and 5b show that the strain with a knockout for the SRX1 gene exhibits hypersensitivity to peroxide.

FIG. 6 also shows that the Srx1 protein is necessary for resistance against the peroxide stress.

In particular, this FIG. 6 shows that the overexpression of TSA1 completely corrects the resistance deficiency of the Δsrx1 strain, showing that this sensitivity is due to a deficiency in peroxidase activity. The overexpression of SRX1 in a Δtsa1 yeast has no effect, unlike the same overexpression in a wild-type strain. This shows that the presence of Tsa1 is essential for Srx1 function.

SRX1 gene function is linked to the TSA1 gene. The overexpression of TSA1 restores the deficiency of tolerance to H₂O₂ and to t-BOOH in the Δtsa1 strain, but overexpression of the SRX1 gene does not cause any effect of this type in the Δtsa1 strain, although it slightly increases the tolerance of the wild-type strain to t-BOOH. These data indicate that Srx1 acts via Tsa1, while the overexpression of Tsa1 can compensate for a deficiency in Srx1 protein.

The substitution of Cys84 to serine (Srx1C^{ys84S}) completely eliminates the function of Srx1 in hydrogen peroxide tolerance (FIG. 5a) and the formation of an Srx1-Tsa1 disulfide bridge, indicating that this binding is essential for the function of Srx1 and is due to Cys84.

### EXAMPLE 5

### ATP is Necessary to Reduce the Cys-SO₂H Form of Tsa1

5.1 Materials and Methods

See example 1.

5.2 Results

In order to study in greater detail the reduction of the Cys-SO₂H form of Tsa1 by Srx1, the recombinant Srx1 protein expressed by a baculovirus was produced. It shows that purified Srx1 allows reduction of the SO₂H form of purified Tsa1, and that this reduction takes place only in the presence of ATP and of lysates from wild-type cells (FIG. 8). These data show that Srx1 catalyzes the reduction of the sulfonate form of Tsa1.

In fact, the Srx1 protein allows the reduction of the Cys-SO₂H form of the Tsa1 protein present in the lysates of ΔSrx1 cells treated with H₂O₂ in a dose-dependent manner, only when ATP is added (FIGS. 8a and b). GTP and AMP-PNP, which is a non-hydrolyzable ATP homolog, have no effect on the catalysis. The addition of EDTA to the lysate inhibits the Srx1-dependent reduction of Tsa1, and the reintroduction of Mg⁺⁺ or of Mn⁺⁺, but not of Fe⁺⁺, Ca⁺⁺, Cu⁺⁺ or Zn⁺⁺, restores the reduction. Finally, purified Srx1 completely reduces the purified and oxidized Tsa1 form in vitro in the presence of ATP, of Mg⁺⁺ or of Mn⁺⁺ and of DTT (FIG. 8c), demonstrating that Srx1 itself catalyzes the reduction of the Cys-SO₂ form to the Cys-SH form. The coupling of ATP hydrolysis and the specific need for Mg⁺⁺ or Mn⁺⁺ greatly suggest that substrate phosphorylation is carried out by Srx1, as a step in the process for reducing Cys-SO₂H, although an intermediate has not yet been detected, probably because of the highly unstable nature thereof. The disulfide bond between Srx1 and Tsa1 also suggests that a mechanism that functions on the basis of a thiol group exists as another step in this process. The activity of Srx1 mutants was tested by substituting each of its 3 cysteines. The substitution of Cys84 (Srx1^{Cys84S}), which is conserved among the Srx1 homologs in other eukaryotes, completely eliminates the formation of the disulfide bridge between Srx1 and Tsa1 and the reduction of the Cys-SO₂H form of Tsa1, whereas the other cysteine mutants have no effect for Srx1^{Cys106S} or a minor effect for Srx1^{Cys48S}. These data indicate that the Srx1-Tsa1 bond originates from Cys84 of Srx1 and that it is essential for the Srx1-mediated reduction of Tsa Cys-SO₂H. The substitution of Cys84 to serine also eliminates the role of Srx1 in vivo in hydrogen peroxide tolerance, indicating furthermore that the Srx1-dependent reduction of Tsa1 Cys-SO₂H is important in order for the peroxidase to function.

The sulfinic acid of the cysteines in proteins cannot be reduced by monothiol or dithiol reducing agents.

The following mechanism of action is proposed:

Sulfiredoxin catalyzes this reduction according to a multistep process by acting both as a specific phosphotransferase and as a thioltransferase (FIG. 8). Reduction of the sulfinic acid of the cysteine probably requires its initial activation, which can be carried out by formation of a phosphorylated sulfinic ester, as the need for ATP and for Mg⁺⁺ indicates. This modification allows the sulfide residue to be attacked by the cysteine at the activated site of Srx1, and then the temporary formation of an intermolecular thiolsulfinate between Srx1 and Tsa1. The thiolsulfinate exists during oxidative stress and is accessible to thiol-dependent reduction. Thus, once formed, the thiolsulfinate between Srx1 and Tsa1 is converted to two Cys-SH by successive thiol-redox exchanges initially involving the reductive cleavage of the thiolsulfinate bridge to a sulfenate and a disulfide bridge by virtue of the electrons provided by DTT in vitro, and probably by thiolredoxin in vivo.

### EXAMPLE 6

### Identification of Human Sulfiredoxin (hSrx1) and Demonstration of its Catalytic Activity

6.1 Materials and Methods

The hSrx gene (SEQ ID No. 4) was cloned by PCR from cDNA prepared by reverse transcription from cells of a human tumor line MCF-7, using the oligonucleotides:

(SEQ ID No. 13)
TTAATTGAATTCATGGGGCTGCGTGCAGGAGG
and
(SEQ ID No. 14)
TTTTCCTTTTGCGGCCGCCTACTACTGCAAGTCTGGTGTGGATG.

The hSrx1 coding sequence was cloned into the vector pFastBac1 (Invitrogen) and then expressed in High Five insect cells (see example 1, point 1.4).

The lysate of High Five cells overexpressing hSrx1 was used, in vitro, to test its activity for reducing the human peroxyredoxins Prx1 and Prx2 superoxidized in the sulfinic acid form (FIG. 9). 6HIS-Prx1 and 6HIS-Prx2 were expressed, purified and superoxidized according to the same method as Tsa1 in S. cerevisiae. The protocol and the method are identical to those of example 1 (points 1.3 and 1.4).

6.2 Results

FIG. 9 illustrates the results obtained and shows the ability of hSrx1, expressed from Baculovirus in High Five cells, to reduce the human peroxyredoxins 6His-Prx1 and 6His-Prx2 superoxidized in the cysteine sulfinic acid form. This reduction requires the presence of the cofactors ATP (1 mM) and Mg⁺⁺ (1 mM) and dithiothreitol (2 mM).

The Baculovirus extracts express either hSrx1 (h Srx) or the Tau138 protein (control). The method and the protocol of this experiment are identical to those specified in example 5.

As emerges from the above, the invention is in no way limited to those of its methods of implementation, execution and application which have just been described more explicitly; on the contrary, it encompasses all the variants thereof that may occur to those skilled in the art, without departing from the context or the scope of the present invention.

1-25. (canceled)

26. A protein, sulfiredoxin (Srx), which comprises at least one catalytic site having a motif: FXGCHR, wherein X is G or S (SEQ ID NO: 15).

27. The protein of claim 26, having a molecular weight of about 8 to 14 kDa.

28. The protein of claim 26, which is a sulfiredoxin of a microorganism, plant or higher organism, which comprises between about 80 and 170 amino acids and at least the one catalytic site having the motif: FXGCHR, wherein X is G or S (SEQ ID NO: 15), and having the following percentage identifies and similarities:
yeast/human: 32% identity and 67% similarity
yeast/plants: 23% identity and 39% similarity
yeast/mouse: 31% identity and 51% similarity
yeast/fungi: 80% identity and 9% similarity.

29. The protein of claim 26, which is selected from proteins having sequences corresponding to SEQ. ID. Nos. 1-10.

30. The protein of claim 26, which is from yeast and is Srx1, having a molecular weight of 13 kDa.

31. The protein of claim 26, which is from humans and is hSrx1, having a molecular weight of 13.6 kDa.

32. A protein which catalyzes reduction of Cys-SO₂H groups.

33. The protein of claim 32, which catalyzes reduction of peroxyredoxin (Prx) in a superoxide (Cys-SO₂H) form to a corresponding thiol form.

34. An isolated peptide corresponding to the catalytic site of Srx as defined in claim 26.

35. A pharmaceutical composition, comprising an amount of a protein comprising a sequence selected from the group consisting of SEQ ID Nos. 1-3 and 5-10 and at least one pharmaceutically acceptable excipient, said protein amount being effective for testing a disorder arising from a defect in a Prx/Srx antioxidizing system in a mammal.

36. A method of screening for disease by evaluating involvement of a Prx/Srx antioxidizing system, which comprises the steps of:
(a) bringing cells of a biological sample into contact, in vitro, with hydrogen peroxide (H₂O₂),
(b) detecting Prx-Cysₚ-SO₂H formed, between about 1 hour and 4 hours after step (1), and
(c) establishing a ratio of amounts of Prx-Cysₚ-SO₂H and of Prx-Cysₚ-SH, from about 4 hours after step (1).

37. The method of claim 36, wherein the disease is cancer.

38. The method of claim 36, wherein the disease is a neurodegenerative disease.

39. The method of claim 36, wherein the disease is aging.

40. A method of screening for disease by genotyping of sulfiredoxin, using total RNA of a biological sample, which comprises the steps of:
(a) extracting the total RNA from the biological sample,
(b) preparing specific sulfiredoxin cDNA by amplification of the RNA using the following two primers:

| | |
|---|---|
| GTCCCGCGGCGGCGGCGACG | (SEQ ID No. 11) |
| AGCAGGTGCCAAGGAGGCTG, | (SEQ ID No. 12) |

these sequences being located, respectively, upstream and downstream of the human sulfiredoxin ORE′ (GenBank No. AAH47707),
(c) establishing its nucleotide sequence, and
(d) comparing with respect to a DNA sequence encoding an Srx protein, as defined above, derived from the same species as that of the biological sample to be analyzed.

41. The method of claim 40, wherein the disease is cancer.

42. The method of claim 40, wherein the disease is a neurodegenerative disease.

43. The method of claim 40, wherein the disease is aging.

44. A method of screening for diseases which entails relative quantification of the mRNA encoding sulfiredoxin from a total cDNA prepared from a human biological sample, by comparison with a reference sample.

45. The method of claim 44, wherein the quantification comprises the steps of:
(a1) preparing cDNA from the total RNA by reverse transcription with appropriate primers, and in particular random hexanucleotide primers;
(a2) amplifying said cDNA in the presence of the pair of primers:

| | |
|---|---|
| GTCCCGCGGCGGCGGCGACG | (SEQ ID No. 11) |
| AGCAGGTGCCM\GGAGGCTG, | (SEQ ID No. 12) |

in the presence of a fluorescent reporter, and simultaneously or sequentially,
(a3) detecting the amount of the amplimer (or amplicon) by measuring the fluorescent signal.

46. The method of claim 45, wherein the disease is cancer.

47. The method of claim 45, wherein the disease is a neurodegenerative disease.

48. The method of claim 45, wherein the disease is aging.

49. The method of claim 45, wherein the fluorescent reporter is selected from the group consisting of agents that bind to double-stranded DNA and fluorescent probes.

50. The method of claim 45, wherein when said fluorescent reporter is a probe, it is selected from the group consisting of the probes defined by the following sequences:
(SEQ ID No. 13)
TTAATTGAATTCATGGGGCTGCGTGCAGGAGG
and
(SEQ ID No. 14)
TTTTCCTTTTGCGGCCGCCTACTACTGCAAGTCTGGTGTGGATG.

51. A method of screening for disease which comprises the steps of:
a) immunodetecting an Srx protein in a biological sample, using an antibody obtained by immunization of an animal with an Srx protein or the peptide FXGCHR, with X=G or S (SEQ ID NO: 15), after separating total proteins by electrophoresis, and then
b) evaluating quality and amount of the Srx protein compared with a control Srx protein.

52. The method of claim 51, wherein the disease is cancer.

53. The method of claim 51, wherein the disease is neurodegenerative disease.

54. The method claim claim 51, wherein the disease is aging.

55. A method of obtaining plants having an increased stress resistance, which comprises evaluating a Prx/Srx antioxidizing system of a plant using the protein of claim 26, and selecting a plant based upon the evaluation.

56. A host cell transformed with a recombinant vector comprising a sequence encoding an Srx protein, defined by a sequence selected from the group consisting of the sequences SEQ ID Nos. 1-3, 5, 6 and 8-10.

57. The host cell of claim 56, which is an S. cerevisiae strain modified with a vector overexpressing the Srx1 gene.

58. The host cell of claim 56, which is a mammalian cell modified with a vector overexpressing the hSrx1 gene.

59. The host cell of claim 56, wherein the vector is an E. coli/S. cerevisiae shuttle vector comprising, at an EcoRI cloning site, a sequence encoding the Srx protein and the promoter of the Srx gene.

60. A method of screening for medicinal products capable of modulating activity of a Prx/Srx antioxidizing system, which comprises the steps of:
(a) bringing a sample substance into contact with the host cells of claim 41, in the presence of hydrogen peroxide,
(b) detecting Prx-Cys formed, between about 1 hour and 4 hours after step 1), and,
(c) establishing a ratio of amounts of Prx-Cys and of Prx-Cys from about 4 hours after step 1).

61. (v) A method of screening for medicinal products for treating a condition arising from a fault in a Prx/Srx antioxidizing system, which comprises the steps of:
a) bringing a sample substance into contact with an extract of the host cells of claim 41, or a biological sample of a nonhuman transgenic animal selected from the group consisting of animals in which the gene of the Srx protein is knocked out and animals in which a gene of the Srx protein is overexpressed, in the presence of hydrogen peroxide,
b) measuring an antioxidizing activity of the Prx/Srx system of the mixture obtained in a), and
c) selecting the substances capable of stimulating or of inhibiting said activity.

62. The method of claim 61, wherein the measurement of said activity is carried out by detecting the Prx-Cysₚ-SO₂H formed, between about 1 hour and 4 hours after said bringing into contact according to step (a), and establishing the ratio of the amounts of Prx-Cysₚ-SO₂H and of Prx-Cysₚ-SH, from about 4 hours after said bringing into contact according to step (a).

63. A method of screening for medicinal products, for treating a condition related to a fault in a Prx/Srx antioxidizing system, which comprises the steps of:
(a) bringing a sample substance into contact with nonhuman transgenic mammals selected from the group consisting of animals in which the gene of the Srx protein is knocked out and animals in which the gene of the Srx protein is overexpressed, and
(b) measuring survival of the animal.

64. Anti-Srx antibodies, obtained by immunization of an animal with an Srx protein defined by a sequence selected from the group consisting of the sequences SEQ ID No. 1-3, 5, 6 and 8-10 or the peptide FXGCHR, with X=S (SEQ ID NO: 16), as claimed in claim 34.

65. The anti-Srx antibodies of claim 64, which are monoclonal antibodies.

66. The anti-Srx antibodies of claim 64, which are polyclonal antibodies.

67. A method of reducing a product comprising at least two cysteines with redox activity, which comprises the step of bringing said protein into contact with a sulfiredoxin (Srx), as defined in claim 26, which comprises at least one catalytic site having the following motif: FXGCHR, with X=G or S (SEQ ID NO: 15), in the presence of ATP and magnesium.

68. A method of synthesizing a product comprising Cys-SH residues from products comprising Cys-SO₂H residues, which comprises the step of reducing the product comprising the Cys-SO₂H residues to a product comprising Cys-SH residues, in the presence of a sulfiredoxin as defined in claim 26, ATP and magnesium.
